(19) United States
(12) Patent Application Publication
(10) Pub. No.: US 2021/0322350 A1
(43) Pub. Date: Oct. 21, 2021

(54) EXOGENOUS KETONE SUPPLEMENTATION COMPOSITIONS, SYSTEMS AND METHODS

(71) Applicant: Ketone Clarity LLC, Sheridan, WY (US)

(72) Inventors: James Christopher Schroeder, San Carlos, CA (US); Heidi Heather Hynes, San Carlos, CA (US)

(21) Appl. No.: 17/002,691

(22) Filed: Aug. 25, 2020

Related U.S. Application Data
(60) Provisional application No. 63/013,340, filed on Apr. 21, 2020.

(51) Int. Cl. A61K 31/191; A23L 33/00; A23L 29/00

(52) U.S. Cl. CPC A61K 31/191; A23L 29/035; A23L 33/30

(57) ABSTRACT

The claimed invention addresses the benefits of exogenous ketone supplementation in a subject in need thereof. In particular, the exogenous ketone supplementation includes the natural D-Beta HydroxyButyric acid, also described as natural D-BHB. As detailed in representative FIG. 1, natural D-BHB is preferentially consumed in the brain to glucose and additionally has a wide range of positive regulatory effects on the body and mind. Consumer applications for supplementation with natural D-BHB include improved sleep, anti-hunger, anti-anxiety, improved cognitive enhancement and athletic duration and post workout recovery through consumption of 10 grams or more of natural D-BHB. Therapeutic applications for exogenous natural D-BHB supplementation include anti-cancer, anti-kidney disease, anti-cognitive disorders and improved viral resistance through consumption of 10-20 grams or more of D-Beta HydroxyButyric acid.

### BACKGROUND OF THE INVENTION

### Technical Field

The claimed invention has applicability in consumer health supplementation. With greater particularity, the claimed invention is relevant to consumer wellness management while undertaking the ketogenic diet. With still greater particularity, the claimed invention is directed at novel uses of the ketone body D-HydroxyButyric Acid along with related systems and methods.

### Background Art

Contrary to traditional dietary views, unlimited glucose and fructose in the human diet does not lead to optimum health and wellness. With Type II diabetes on the rise and all time high number of childhood obesity cases, it is becoming all too clear that the old adage “a calorie is just a calorie regardless of source” is simply just not true. The metabolic health challenges facing glucose awash individuals include high blood pressure, excess blood sugar, diabetes, atherosclerosis and liver and kidney problems. More importantly, the causal connection between excess dietary sugar and Central Nervous System (CNS) conditions is only beginning to be characterized. Related conditions including Alzheimer's (now being considered as Type III diabetes) and Parkinson's are likely to have a direct correlation involving metabolic breakdown and energy insufficiency owing to an overload of bodily systems unable to process excess sugar.

Excess sugar in the human diet often presents multiple and directly inter-related symptoms and maladies. Excess dietary sugar often leads to pre-diabetic or full Type II diabetes conditions. Diabetes damages arteries and makes them targets for hardening, called atherosclerosis. That can cause high blood pressure, which if not treated, can lead to trouble including blood vessel damage, heart attack, and kidney failure. Moreover, high blood pressure can have a causal link to many brain related maladies as well. With respect to cancer growth and particular tumors, the link between glucose consumption and unrestricted tumor growth is clear.

Controlling diet is one known way to address excess glucose intake and related consequences. Following the ketogenic diet, however, requires very strict regulation and control of carbohydrates and sugar along with high fat intake. Owing to the difficult dietary regimen, attempts to follow the ketogenic diet more often than not fail.

To date, exogenous ketone supplementation has been attempted as an aide in following the ketogenic diet. Unfortunately, ketone salts result in an abnormally high salt intake rate and the newly created (R)-3-hydroxybutyl (R)-3-hydroxybutyrate (D-β-hydroxybutyrate ester) or ‘ketone ester’ is a never before seen in man synthetic creation with bioavailability and other long term concerns.

Consequently, despite the high popularity of the ketogenic diet there has been a pressing and long term demand for new ketogenic supplementation alternatives.

### BRIEF SUMMARY OF THE INVENTION

In answer to the need of alternative aids to ketone supplementation, the claimed invention utilizes novel exogenous ketone compositions, systems and related methods of first impression. Novel compositions, systems and methods that incorporate the natural ketone D-Beta-HydroxyButyric Acid in dietary supplementation and therapeutic delivery forms are hereby disclosed and claimed.

By supplementing an individual with exogenous ketone preparations incorporating the natural D-Beta-HydroxyButyric Acid (D-BHB), internal body ketone levels rapidly rise when directly compared with non-supplementing individuals. The ketone body D-BHB is first and foremost an energy source, which is directly and preferentially metabolized by the heart, kidneys and brain. D-BHB is also active as a signaling mechanism, resulting in broad and beneficial health related benefits.

In healthy individuals, consuming D-BHB as a nutritional supplement provides near term energy for active sports and endurance activities. Owing to its unique metabolic properties, often a reduced need for oxygen has been clearly demonstrated in the example of swimmers requiring fewer instances of surfacing to take a breath when compared with similar but non-supplemented athletes. Physiologically, blood pressure and blood glucose are often reduced in a dose dependent manner. Cognitively, improved concentration is often attained after exogenous D-BHB supplementation. As a signaling mechanism it has a direct and long lasting effect on bio-regulatory mechanisms including hunger and sleep. D-BHB has a complex yet beneficial interplay with the hunger hormone ghrelin, providing a dual role suppressing hunger in sedentary individuals while simultaneously stimulating appetite in high performance athletes requiring additional nourishment to sustain excessively strenuous activities. As an archaic energy source, D-BHB is also believed to positively shape the microbiome of the healthy individual as well as increased male fertility.

In a preferred embodiment, a healthy individual consumes 10-20 g/day of exogenous ketone D-Beta-HydroxyButyric Acid alone or in combination with a ketogenic diet. In a preferred system and related method, blood ketone and glucose levels are measured by a simple formula of (Glucose mmol/L)/(Ketone mmol/L) to generate a mathematical score on the “Glucose/Ketone Index, or GKI. The GKI can be utilized to determine healthy ketone generation and guide positive effects. When preferred embodiments are followed according to the claimed invention, enhanced ketone and improved GKI scores are attained when compared to pre-existing known methods including unaided fasting, racemic ketone salts and the newly created synthetic “ketone ester.”

In terms of therapeutic benefit for existing diseases, the claimed invention provides significant benefits over traditional pharmacological treatments. By utilizing the claimed compound, system and method a Glucose/Ketone Index of 1 or less readily attainable through regular D-BHB consumption. While debate continues regarding the metabolic vs genetic origin of cancer, it is clear that certain cancers thrive when utilizing glucose as a food source. Utilizing the principle known as the “Warburg Effect”, new approaches to cancer management are now being considered. When the GKI is below a level of one, ketones dominate over glucose and create the opportunity to starve specific types of tumor cells in situ. With greater specificity, brain, breast, kidney and pancreatic cancers may be specifically vulnerable to D-BHB supplementation as a therapeutic approach. In additions, fibroid conditions may be therapeutically alleviated in the kidneys, uterus and other bodily areas.

As an energy source, D-BHB offers therapeutic benefit particularly in the human heart where it is preferentially consumed as an energy source over glucose and preferentially to other organs of the body. While current interest exists on ketone bodies as neuroprotectives, the claimed D-BHB composition offers higher energy access than ketone salts and quicker absorption when compared to the known synthetic “ketone ester” products. Consequently, therapeutic benefits against physical brain trauma along with neuroprotection and neuroregeneration are both clear and uniquely compelling. It is important to characterize the benefits as nutritional rather than pharmacological. Owing to its protective role involving Reactive Oxygen Species (ROS), D-BHB has demonstrated radiation protection offering unique protection in radioactive environments on the ground as well as in space. As the compound is smaller than 500 Daltons, it can directly pass through the epidermal layer of the skin, offering skin nutrition and protection. In a related and foreseeable microenvironment application, reduction of gout in joint areas has been achieved with modest levels of D-BHB consumption.

From an energy as well as a neurochemistry perspective, D-BHB offers enhanced energy for the brain to alleviate ageing related conditions including Alzheimer's and Parkinson's disease. While applications such as epilepsy and anti-seizure are known, this is the first time the natural form of D-BHB has been made available as a metabolically direct exogenous supplement. As an extension, regulation of related brain conditions including depression and anxiety are readily foreseeable embodiments of the claimed invention along with utility as a mechanism for cessation of long term anti-depression medication use.

As a regulatory mechanism, exogenous D-BHB has a benefit not only in glucose and insulin management in Type II diabetic and pre-diabetic conditions, bone protective benefits occur with direct osteoclast regulation. The role of D-BHB in reducing inflammation and energy replenishment make it uniquely compelling for therapeutic benefits against Multiple Sclerosis as well as orphan diseases including MADD, Huntington's Disease and Angelmann's syndrome.

### BRIEF DESCRIPTION OF THE SEVERAL VIEWS OF THE DRAWING(S)

The accompanying drawings are included to better illustrate exemplary embodiments of the claimed invention.

FIG. 1 is a graphical illustration of endogenous ketone blood levels in the brain.

FIG. 2 is a structural illustration of endogenous ketones.

FIG. 3 is a graphical illustration of endogenous ketone levels by age.

FIG. 4 is a structural illustration of chemical structures of D-BHB, salt and ketone ester.

FIG. 5 is a graphical illustration of exogenous ketone production by three precursors.

FIG. 6 is a graphical illustration of exogenous ketone blood levels by three precursors.

FIG. 7 is a graphical illustration of D-BHB exogenous ketone blood levels.

FIG. 8 is a schematic illustration of the Glucose/Ketone Index.

FIG. 9 is a graphical illustration of blood levels of ketones and glucose resulting from the claimed invention.

FIG. 10 is a graphical illustration of GKI calculations based upon blood levels of ketones and glucose resulting from the claimed invention.

FIG. 11 is a graphical illustration of improved sleep time resulting from enhanced D-BHB levels according to the claimed invention.

FIG. 12 is a graphical illustration of improved blood pressure resulting from enhanced D-BHB levels according to the claimed invention.

FIG. 13 is a graphical illustration of enhanced blood oxygen resulting from enhanced D-BHB levels according to the claimed invention.

FIG. 14 is a graphical illustration of lowered blood glucose resulting from enhanced D-BHB levels for Type II diabetic and pre-diabetic conditions according to the claimed invention.

FIG. 15 is a graphical illustration of lowered hunger resulting from enhanced D-BHB levels according to the claimed invention.

FIG. 16 is a graphical illustration of improved physical endurance resulting from enhanced D-BHB levels according to the claimed invention.

FIG. 17 is a graphical illustration of improved mental concentration resulting from enhanced D-BHB levels according to the claimed invention.

FIG. 18 is a graphical illustration of improved mental anti-depression and anti-anxiety activity resulting from enhanced D-BHB levels according to the claimed invention.

FIG. 19 is a graphical illustration of improved neural stimulation and brain protection resulting from enhanced D-BHB levels.

FIG. 20 is a graphical illustration of sustained bone protection resulting from enhanced D-BHB levels according to the claimed invention.

FIG. 21 is a graphical illustration of gout reduction resulting from enhanced D-BHB levels according to the claimed invention.

FIG. 22 is a graphical illustration of heart, kidney and brain metabolism of D-BHB.

FIG. 23 is a schematic illustration of the protective role involving Reactive Oxygen Species (ROS) metabolism of D-BHB for radiation protection on ground and in Space.

FIG. 24 is a schematic illustration of the protective role of D-BHB on the skin.

FIG. 25 is a schematic illustration of the therapeutic role of D-BHB against cancer.

FIG. 26 is a schematic illustration of the therapeutic role of D-BHB against Alzheimer's & Parkinson's diseases.

FIG. 27 is a schematic illustration of the therapeutic role of D-BHB against kidney disease.

FIG. 28 is a schematic illustration of the therapeutic role of D-BHB against Multiple Sclerosis.

FIG. 29 is a schematic illustration of the therapeutic role of D-BHB and microbiome.

FIG. 30 is a schematic illustration of the therapeutic role of D-BHB and antiviral protection.

FIG. 31 is a schematic illustration of a preferred system embodiment according to the claimed invention.

FIG. 32 is a schematic illustration of a preferred method embodiment according to the claimed invention.

### DETAILED DESCRIPTION OF THE INVENTION

The claimed invention addresses novel applications of exogenous supplementation of the natural version of the ketone D-Beta Hydroxybutyric acid to attain enhanced metabolic, neurological and therapeutic benefits. While alternative ketone supplementation methods do exist, they are easily distinguishable owing to their synthetic nature or salt composition characteristics. To best understand the nature and applications of the claimed invention, an overview and introduction to endogenous and exogenous ketone uses are hereby provided.

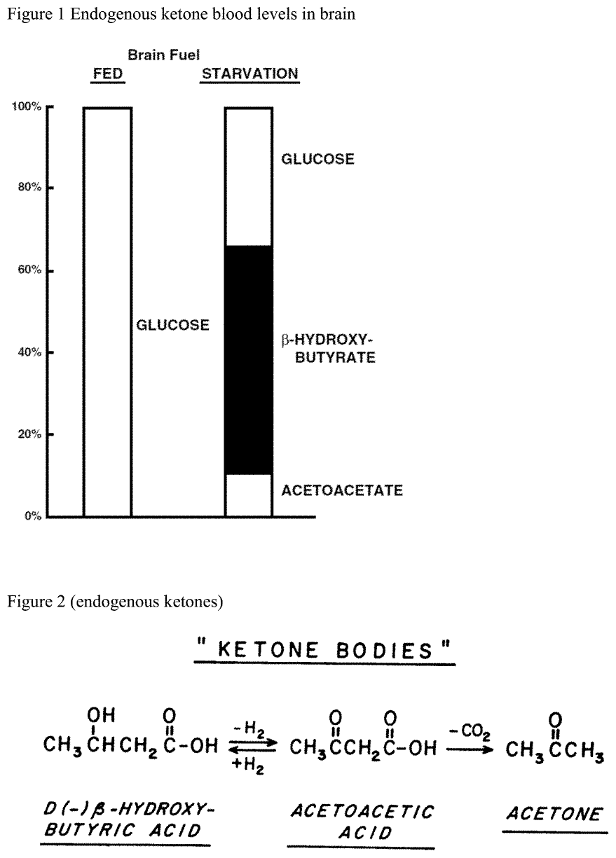

Ketones are naturally generated by the human body during times of nutritional distress and intentional caloric restriction and fasting. FIG. 1 is a graphical illustration of endogenous ketone blood levels in the brain. As depicted in FIG. 1, the naturally produced endogenous ketone is preferentially utilized in the human brain. As a direct and intended consequence of D-B-HydroxyButyric acid (D-BHB) utilization, energy in the brain is increased when D-BHB is naturally created and present in the bloodstream.

FIG. 2 is a structural illustration of endogenous ketones. From a chemical perspective, D-B-HydroxyButyric acid is a metabolic energy source which is widely metabolized throughout the human body. While acetoacetic acid and acetone are also ketone bodies and also produced endogenously, neither provides an equivalent metabolic energy benefit. In addition, neither provides equivalent metabolic signaling benefits in accordance with the claimed invention.

The claimed invention is the first exogenous supplementation of the natural form of D-Beta-Hydroxybutyric acid and as such is chemically and metabolically distinguishable from presently commercially available ketone supplements utilizing a chemical synthetic ‘ketone ester’ or chemically distinct ‘ketone salt’ formulation as neither the ‘ketone ester’ nor the ‘ketone salt’ is naturally produced in the human body.

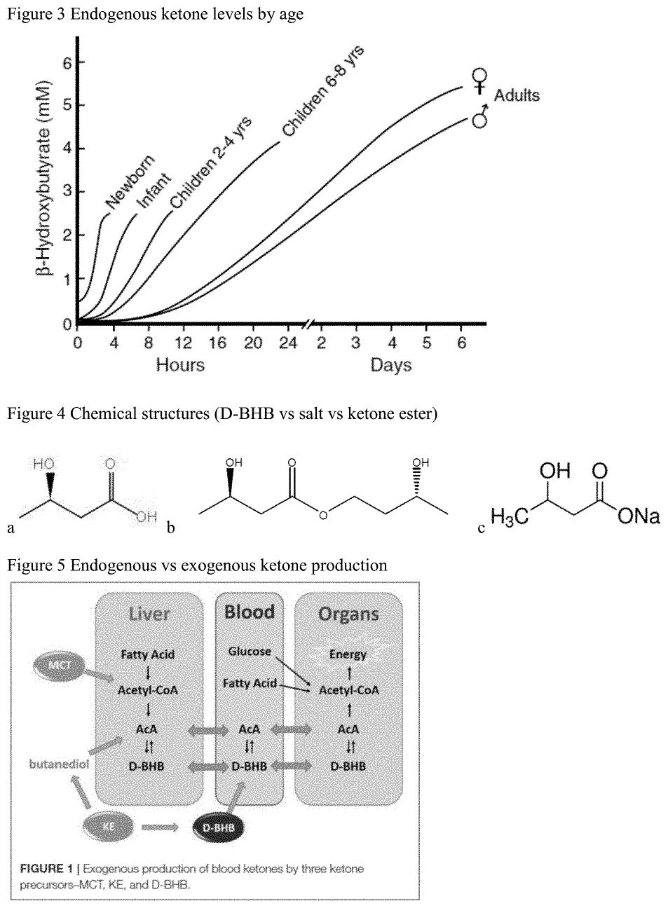

Endogenous production of ketones in the human body varies widely and largely is dependent upon the age of the subject in need thereof. FIG. 3 is a graphical illustration of endogenous ketone levels by age. As can clearly be seen from the graphical illustration, younger individuals are more readily and more often in the metabolic state of ketosis wherein the blood levels are over 1.5 mmol/L of D-Beta-Hydroxy-Butyric acid. A helpful frame of reference to consider D-BHB benefits include a consideration of higher energy states naturally present in young children as a direct metabolic consequence of higher ketone bioavailability.

FIG. 4 is a structural illustration of chemical structures of D-Beta-Hydroxy-Butyric acid, ‘ketone salt’ and ‘ketone ester’ products. As previously mentioned, the claimed invention is based upon novel applications of the natural form of D-B-HydroxyButyric acid administered exogenously. The claimed invention is chemically and metabolically distinguishable from the artificial and chemically synthetic ‘ketone ester’ products which are marketing the synthetic (R)-3-hydroxybutyl (R)-3-hydroxybutyrate (D-β-hydroxybutyrate ester) as a nutritional supplement alone or combined with ‘ketone salt’ products. The ‘ketone salt’ products presently available have a salt component to them such as sodium, calcium or potassium. In terms of chemical composition and bioavailability, ‘ketone salt’ products are commonly racemic in nature and often contain both the ‘D’ and ‘L’ (or ‘R’ and ‘S’) forms of Beta-Hydroxybutyrate. Ketone salts, consequently, are often poorly metabolized owing to the limited utilization in the human body of the L (or ‘S’) form.

Regarding Bioavailability: utilizing D-Beta-HydroxyButyric acid according to the claimed invention is distinguishable from currently available commercial ketone supplements in a number of ways, not the least of which is when the supplement becomes available to a subject in need thereof. FIG. 5 is a graphical illustration of exogenous ketone production by three precursors. The so called ‘ketone ester’ products are synthetic and never before seen in nature or humans and as such are not bioavailable as purchased and consumed. The first component of the synthetic ‘ketone ester’ becomes D-Beta-hydroxybutyrate upon digestion in the stomach with the second portion becoming bioavailable after processing in the liver. Ketone salts similarly require similar precursor processing to disassociate from the salt component of the chemical compound. For illustrative purposes, Medium Chain Triglycerides (MCT) are also often consumed to generate ketones endogenously and are similarly processed in the liver before being metabolized into D-BHB.

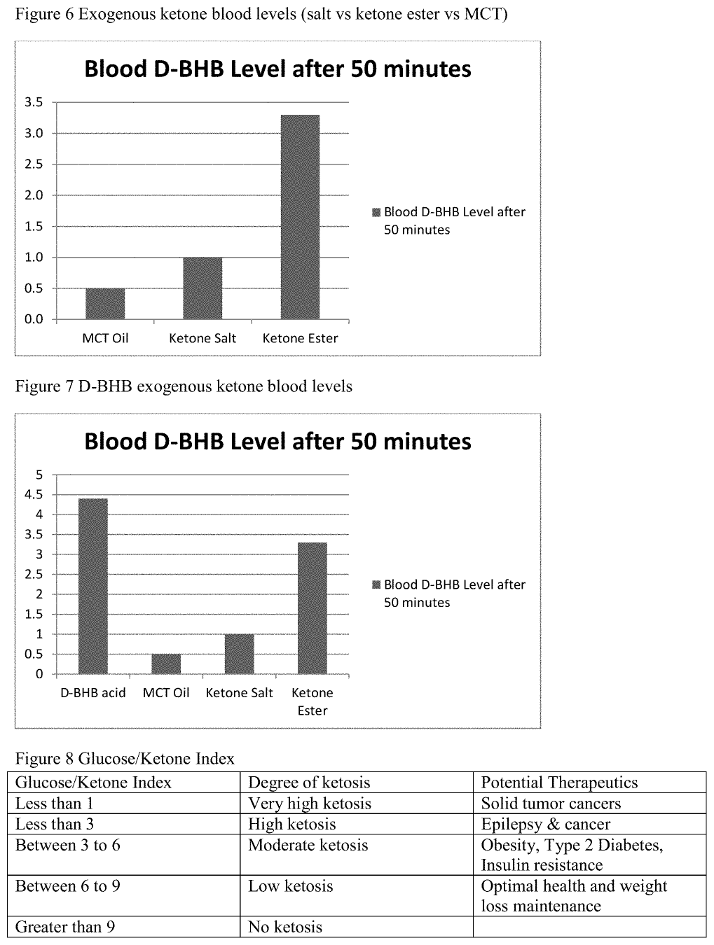

Technical effect: raising blood ketone levels by currently known methods. FIG. 6 is a graphical illustration of exogenous ketone blood levels by three precursors using Medium Chain Triglycerides, ketone salts and ketone esters. Traditionally, as illustrated in FIG. 2, the human body naturally generates ketones by fasting or by following the ‘ketogenic diet’ in which fats and protein are primarily consumed in the absence or minimal presence of carbohydrates. In the healthy adult, blood ketone levels can be naturally raised from a 0.0 mmol/L baseline to 0.5 mmol/L while following a normal diet. FIG. 6 demonstrates the ability of commonly available exogenous ketone precursors and dietary ketone supplements to raise blood ketone levels over time.

While MCT oil can raise blood ketone levels for a long duration, often the rise in ketone levels is nominal at best with a 0.5 mmol/L increase. Ketone salts can raise blood ketone levels to 1.5 mmol/L, but often do so with a metabolic load of heavy salt ingestion along with an undesired racemic mixture of both ‘D’ and ‘L’ isomers of BHB. The so called “ketone ester” synthetic products can raise blood ketone levels to 2-4 mmol/L when consumed at 30-35 g of product but the result is often short lasting.

FIG. 7 is a graphical illustration of D-BHB exogenous ketone blood levels. In contrast to existing methods, consumption of 10-30 g of exogenous D-Beta-HydroxyButyric acid according to the claimed invention results in higher blood ketone levels with prolonged health benefits.

Introducing GKI: Measuring Glucose vs Ketones. FIG. 8 is a schematic illustration of the Glucose/Ketone Index. When measuring both blood ketone levels and blood glucose levels, taking reference from the Glucose/Ketone Index (GKI) can help guide a user to achieve therapeutic benefits according to the claimed invention. The Glucose/Ketone Index is measured by dividing the blood level of glucose by the blood level of ketones measured in mmol/L. By way of illustration, a healthy individual not following the ketogenic diet could have glucose levels of 5.0 mmol/L and ketone levels of 0.1 mmol/L. According to the GKI they would have a value of greater than 9 and as such would not be in ketosis. An individual following the ketogenic diet and potentially supplementing their diet with ketone salts or MCT oil may have a glucose level of 5.6 mmol/L and ketone level of 1.3 mmol/L, resulting in a GKI of 4.3. This moderate ketosis level can be useful for managing obesity and diabetes.

Fasting is the only therapeutic measure against epilepsy recorded in the Hippocratic collection. In the fifth century BC, Hippocrates reported on a man who had been seized by epileptic convulsions. Complete abstinence from food and drink was prescribed, and the cure was effective. Fasting, ketogenic diet and ketone supplementation can also aid fasting in the modern age as well, particularly if the individual is able to achieve high ketosis as indicated by less than 3 on the Glucose/Ketone Index. The primary challenge with generating ketones internally, or endogenously, through controlled fasting or utilization of the ketogenic diet is the lack of long term viability of fasting as well as the high fat requirement coupled with low to no carbohydrate intake of the ketogenic diet. There is a long felt and compelling need for additional ways to generate high ketone levels while lowering corresponding levels of glucose in the human body.

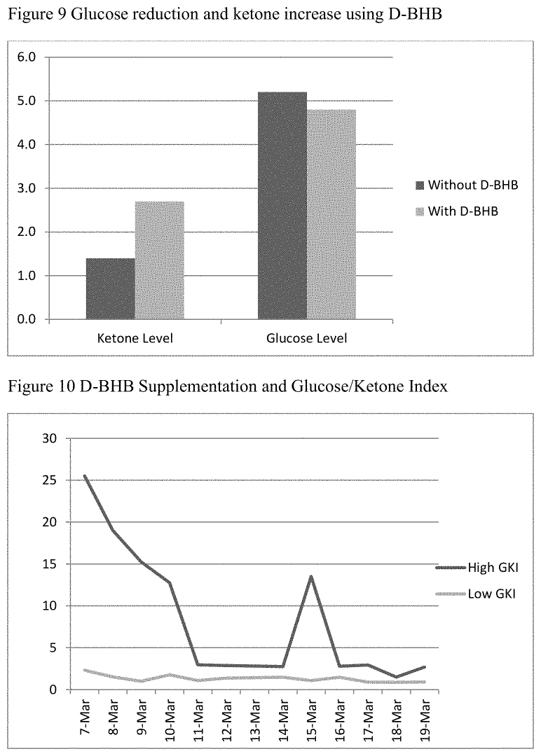

FIG. 9 is a graphical illustration of blood levels of ketones and glucose resulting from the claimed invention. In the graphical illustration, body ketone levels increase from 1.4 mmol/L to 2.7 mmol/L after 50 minutes of consuming 10 g of D-BHB. While ketone levels increase, glucose levels are decreased from 5.2 mmol/L to 4.8 mmol/L. While blood ketone rates can change rapidly as ketones are consumed through exercise or metabolic activity, the lowering of glucose often happens over a prolonged period of time. Consequently, this example is a good introduction to the regulatory effects of the ketone body D-BHB which are independent of and often outlast the energy benefits provided by exogenous ketones even after they are consumed by the body. In this example the subject had a Glucose/Ketone Index (or GKI) of 1.8, indicative of high ketosis and suitable for the attenuation of many neurological conditions.

FIG. 10 is a graphical illustration of GKI calculations based upon blood levels of ketones and glucose resulting from the claimed invention. FIG. 10 illustrates the ‘ramp period’ the human body often goes through when first consuming exogenous ketone supplement according to the invention as claimed. While GKI levels ranging from 1-3 can be obtained with regular D-BHB consumption, often the first several days result in a period of metabolic adaptation while glucose levels subside and ketone levels are increased.

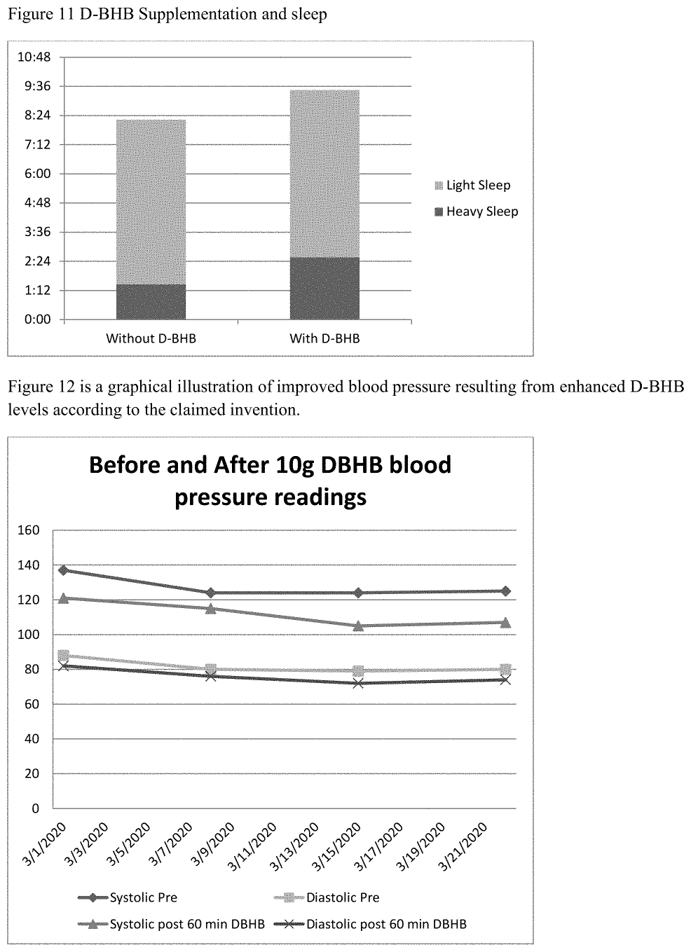

While D-BHB is not a sedative and does not have CNS depressive effects, enhanced deep sleep is a surprising and unintended beneficial result of the claimed invention. During deep sleep, a variety of functions take place in the mind and body. During deep sleep not only does physical recovery occur. The brain recuperates as well during which memories are consolidated and learning and emotions are processed. Physiologically, blood sugar levels and metabolism balance out and the immune system is energized. FIG. 11 is a graphical illustration of improved sleep time resulting from enhanced D-BHB levels according to the claimed invention based on the data detailed in Table 1.

TABLE 1
Sleep data

| | |
|---|---|
| 1:56 | 6:31 |
| 3:01 | 7:52 |
| 2:23 | 6:46 |
| 2:44 | 8:33 |
| 2:25 | 6:17 |
| 1:38 | 6:17 |
| 2:19 | 5:28 |
| 0:58 | 7:41 |
| 1:46 | 6:25 |
| 1:51 | 8:03 |
| 2:32 | 6:27 |
| 2:26 | 5:37 |
| 1:41 | 7:38 |
| 2:28 | 7:32 |
| 3:23 | 5:23 |
| 2:53 | 5:54 |
| 1:49 | 6:27 |

The data gathered in Table 1 reflects unaided sleep cycles in bold together with sleep aided by 10-20 g D-BHB which is reflective of a preferred embodiment of the claimed invention. On average, unaided sleep results in approximately 1.5 hours of deep sleep in an eight hour sleep cycle. In particular, unaided sleep resulted in 1:27 (hours: minutes) of deep sleep along with 6:47 of light sleep. Aided by D-BHB consumption taken prior to sleep, deep sleep often was extended by over an hour without substantially altering light sleep as measured over several sleep cycles. In particular, sleep supplemented with 10-20 g D-BHB resulted in 2:34 of deep sleep along with 6:53 of light sleep. In a preferred and foreseen embodiment of the claimed invention, improved deep sleep is achieved as a result of exogenous ketone D-BHB supplementation.

FIG. 12 is a graphical illustration of improved blood pressure resulting from enhanced D-BHB levels according to the claimed invention. In addition to increasing blood ketones and lowering blood glucose, consumption of exogenous D-BHB according to the claimed invention also has a direct and measurable impact on lowering blood pressure. FIG. 12 demonstrates a measurable decrease in systolic and diastolic blood pressure after ingestion of a moderate 10 g amount of D-Beta HydroxyButyric acid. Blood pressure reduction is measurable within an hour of ingestion and is positive effects of lower blood pressure remain for several hours afterwards. While consumption of 10 g exogenous D-BHB according to the claimed invention is a reasonable baseline level, positive reductions of blood pressure are demonstrated with consumption of larger amounts of D-BHB as well.

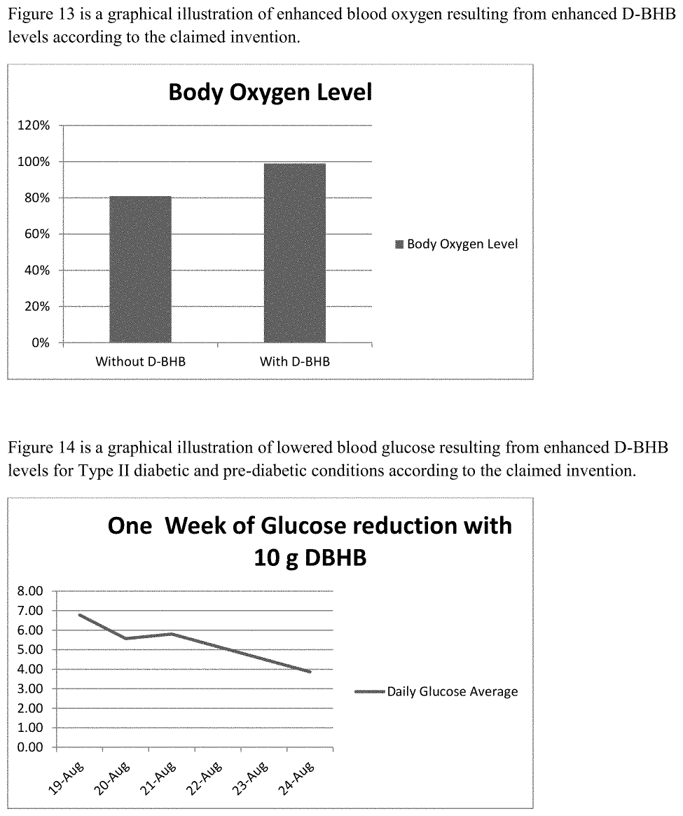

FIG. 13 is a graphical illustration of enhanced blood oxygen resulting from enhanced D-BHB levels according to the claimed invention. As consumption of D-BHB is rapidly metabolized in the body according to the claimed invention, the direct increase of oxygen levels in the blood are correspondingly increased. FIG. 13 demonstrates a representative illustrative example where blood oxygen levels were measured before and 30 minutes after administration of 10 g D-BHB. As blood oxygen levels rapidly increased from 80% to 99%, exogenous D-BHB supplementation with 10 g or more offers a performance sports and therapeutic avenue where benefits from optimized oxygen saturation are desired.

FIG. 14 is a graphical illustration of lowered blood glucose measured in mmol/L as a result of daily 10 g consumption resulting from enhanced D-BHB levels according to the claimed invention. Diet plays a key role in the onset and progression of Type II diabetes and pre-diabetic conditions. The hallmark indication of the progression of this disease is identified through measurement of blood glucose levels. Exogenous ketone supplementation with natural D-BHB according to the claimed invention shows a clear and measurable reduction in blood glucose over time. In an illustrative example, measured blood glucose levels were consistently and sustainably reduced from a baseline of over 7 mmol/L to consistent readings of 4 mmol/L or even less by daily consumption of D-BHB. In a preferred embodiment 10-20 g of D-BHB are consumed, but more or less can be utilized in other embodiments of the claimed invention.

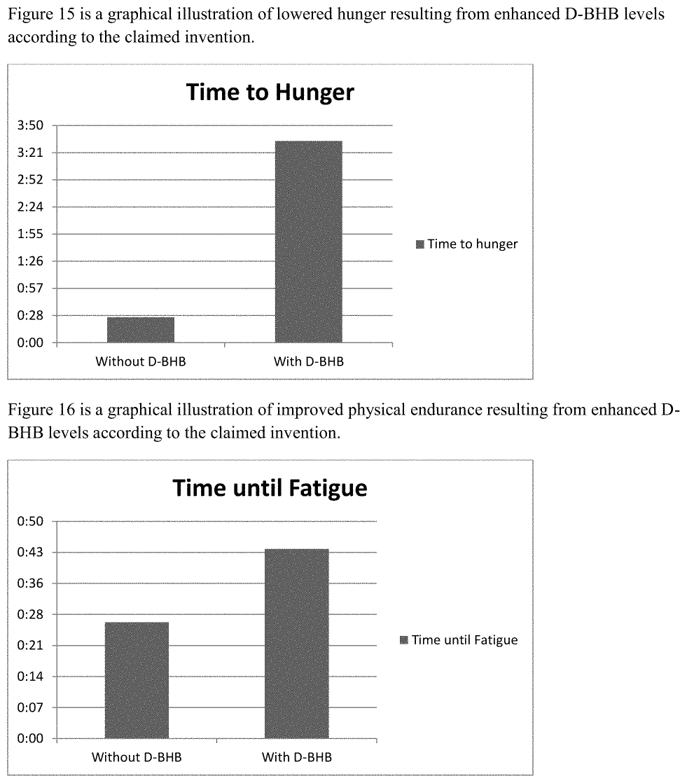

FIG. 15 is a graphical illustration of lowered hunger resulting from enhanced D-BHB levels according to the claimed invention. While the sensation of hunger itself arises from a complex interplay of glucose, insulin and the hormone ghrelin, identification and measurement of hunger is easy and straightforward according to the claimed invention. Hunger is often a key reason why diet attempts such as attempting to follow the ketogenic diet or other diets fail. Hunger is also a significant impediment to individuals attempting to undergo a fasting state for health or other purposes. While even consuming a moderate amount such as 5 g of exogenous natural D-BHB ketone supplement can reduce the sensation of hunger, regular consumption of larger amounts such as 10 g-20 g represent a preferred embodiment of the claimed invention. As demonstrated in FIG. 15, without natural D-BHB ketone supplementation the sensation of hunger can be overpowering within 20-30 minutes of waking. Utilizing the claimed method, however, consumption of natural D-BHB results in greater reduction to even omission of hunger sensations for several hours after rising in the morning. Hunger reduction, however, is provided at any time of day according to the claimed invention and is not just morning dependent.

FIG. 16 is a graphical illustration of improved physical endurance resulting from enhanced D-BHB levels according to the claimed invention. While understanding of the mechanism of ketone enhancement of physical endurance is only beginning, there is a direct and measurable benefit of consumption of natural D-Beta HydroxyButyric acid to enhance physical exertion and post-exercise activity recovery. FIG. 16 illustrates the physical performance parameter in which a subject with and without natural D-BHB ketone supplementation is impeded by shortness of breath and muscle pain and drop in performance owing to lactic acid build-up in the body. Without natural D-BHB ketone supplementation on a measured exercise course pain and fatigue will occur after 27 minutes. With natural D-BHB exogenous ketone supplementation, range of performance is increased to 44 minutes and post-workout recovery time is markedly reduced. While preferred consumption of natural D-BHB can range from 10 g-30 g of natural D-BHB, lesser or greater amounts may also be safely consumed for exercise improvement and benefits.

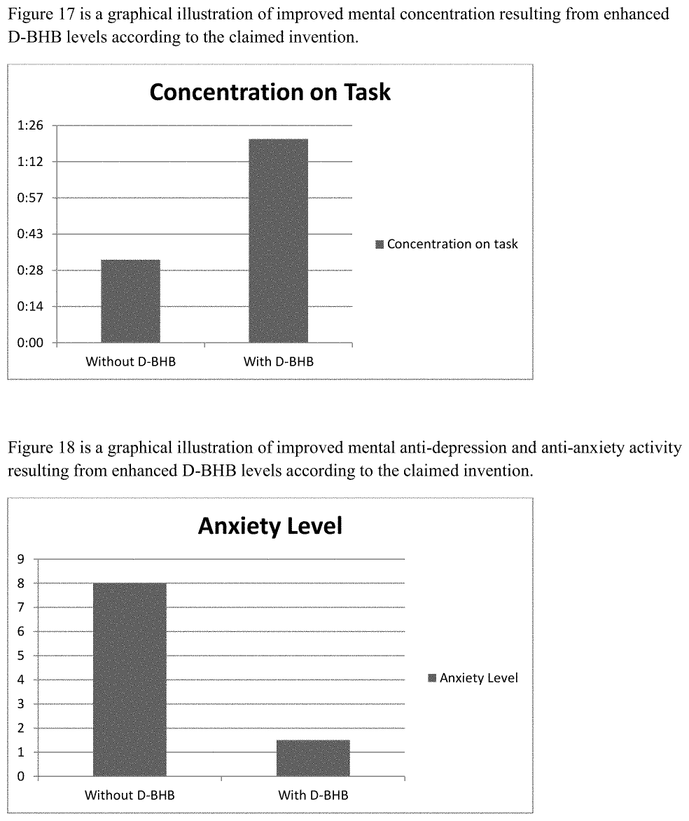

FIG. 17 is a graphical illustration of improved mental concentration resulting from enhanced D-BHB levels according to the claimed invention. As further illustrated by FIG. 1, the natural ketone D-Beta HydroxyButyric acid is a preferred energy source in the brain. FIG. 17 demonstrates the cognitive performance gains attained by nutritional supplementation with the natural D-Beta HydroxyButyric acid. While the mechanism of action is directly attributable to the extra and more direct energy natural D-BHB provides, FIG. 17 illustrates the cognitive ability to focus on task for an extended period of time. While preferred consumption of natural D-BHB can range from 10 g-30 g of natural D-BHB, lesser or greater amounts may also be safely consumed for improved cognitive attention enhancement and benefits. While anti-anxiety benefits are detailed, known variants of the claimed invention include anti-migraine cognitive improvements which are directly attributable to the known lowering of blood pressure as a result of exogenous natural D-BHB supplementation.

FIG. 18 is a graphical illustration of improved mental anti-depression and anti-anxiety activity resulting from enhanced D-BHB levels according to the claimed invention. FIG. 18 demonstrates the cognitive mood enhancement attained by nutritional supplementation with the natural D-Beta HydroxyButyric acid. While the mechanism of action is directly attributable to the role of D-BHB as a signaling metabolite as well as due to the extra and more direct energy natural D-BHB provides, FIG. 18 illustrates the cognitive improvement attained by exogenous D-BHB supplementation in the absence of traditional anti-depressant and/or anti-anxiety medication. While preferred consumption of natural D-BHB can range from 10 g-30 g of natural D-BHB, a best illustrative example reflects at least 10 g exogenous D-BHB supplementation at least twice per day.

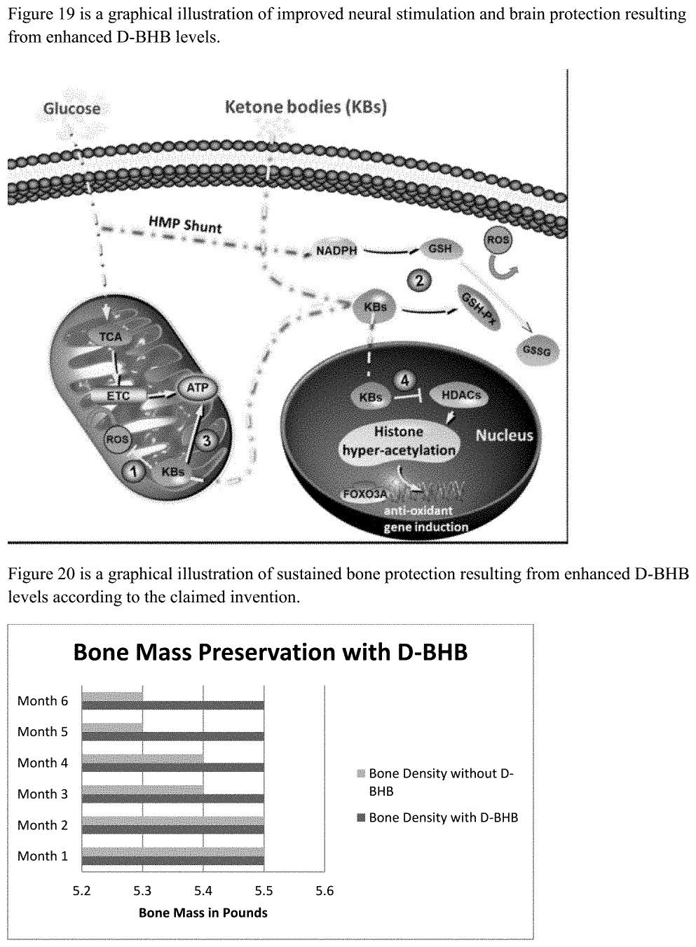

FIG. 19 is a graphical illustration of improved neural stimulation and brain protection resulting from enhanced D-BHB levels. The mechanism of brain protection is well detailed in a recent study entitled “Ketone Bodies in Neurological Diseases: Focus on Neuroprotection and Underlying Mechanisms” (Front. Neurol., 12 Jun. 2019 doi.org/10.3389/fneur.2019.00585). FIG. 19 details the sites of action that underlie in the neuroprotection by D-BHB.1) KBs reduces NAD couple, which decreases ROS production; (2) KBs activate GSH-Px, which enhances ROS elimination; (3) KBs increase ATP concentration; (4) KBs inhibit HDACs, which increases endogenous anti-oxidants.

FIG. 20 is a graphical illustration of sustained bone protection resulting from enhanced D-BHB levels according to the claimed invention. Loss of bone density can be statistically predicted and measured during specific time of life events such as post-menopause as well as during space missions for astronauts in space. FIG. 20 details actual measurements of bone density during exogenous D-BHB consumption of 10 g daily which demonstrates bone density health and retention for a period during which bone loss was expected. Similar benefits against bone loss for space based applications are indicated and expected with consumption of 20 g of exogenous D-BHB daily.

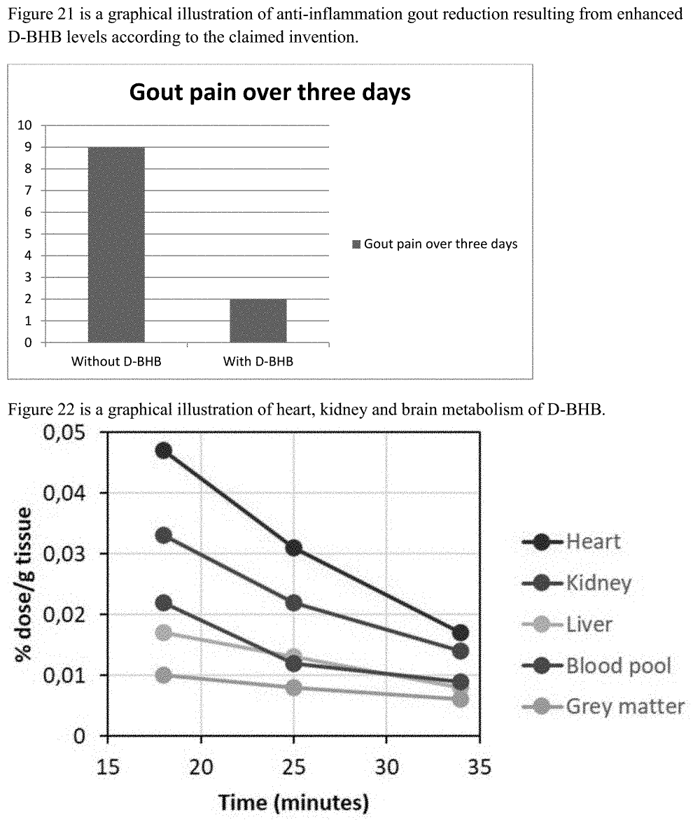

FIG. 21 is a graphical illustration of gout reduction resulting from enhanced D-BHB levels according to the claimed invention. Owing to the role of exogenous natural D-BHB as a regulatory element of the NLRP3 inflammasome, inflammations are reduced as a direct result of exogenous D-BHB supplementation. In a preferred embodiment, 10 g or more of D-Beta HydroxyButyric acid is consumed daily to reduce gout flare-ups. In the representative example detailed in FIG. 21, consumption of 15 g of exogenous D-BHB according to the claimed invention greatly reduced gout pain from extreme to very moderate in just three days. Continued daily consumption of D-BHB entirely removed gout inflammation in less than one week.

FIG. 22 is a graphical illustration of heart, kidney and brain organ distribution after D-BHB precursor oral intake. In a recent metabolism study of D-BHB precursors (Cuenoud B, Hartweg M, Godin J-P, Croteau E, Maltais M, Castellano C-A, Carpentier A C and Cunnane S C (2020) Metabolism of Exogenous D-Beta-Hydroxybutyrate, an Energy Substrate Avidly Consumed by the Heart and Kidney. Front. Nutr. 7:13. doi: 10.3389/fnut.2020.00013), D-BHB consumption is clearly and avidly indicated in the heart, kidneys and brain. D-BHB consumption is correlative with positive effect as D-BHB precursors have already been shown to have positive effect. As the claimed invention is distinguishable from known precursors owing to the exogenous augmentation of natural D-Beta HydroxyButyric acid, the presentation of the claimed natural D-BHB will only be more quickly available owing to the lack of need for processing by the human body before it is bioavailable. Consequently, the claimed invention offers quicker and more pronounced therapeutic options available to brain, heart and kidney applications.

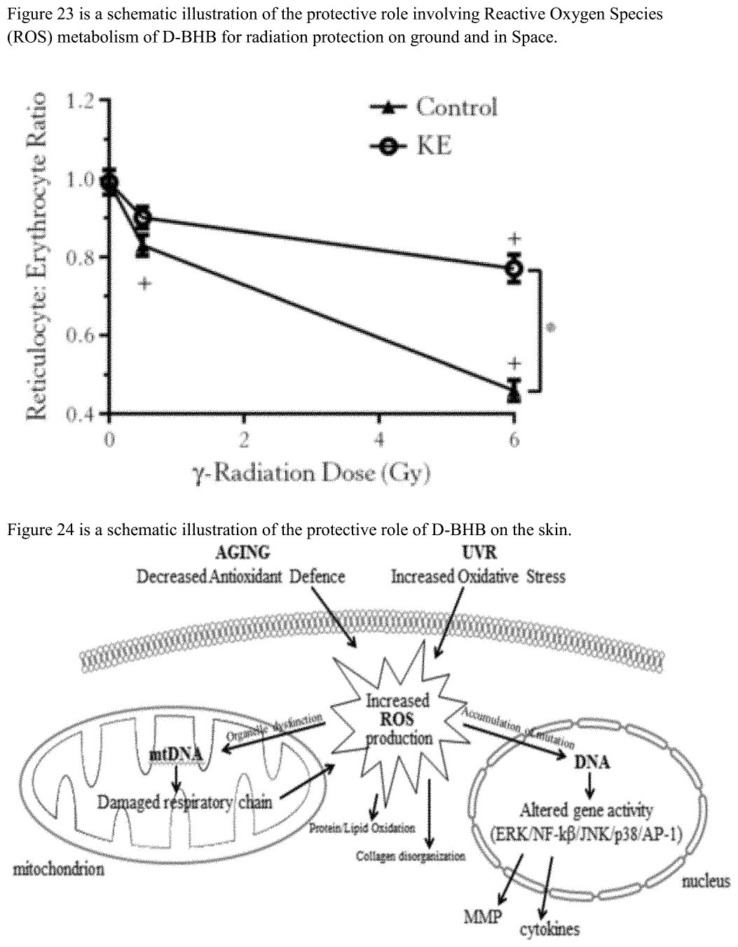

FIG. 23 is a schematic illustration of the protective role involving Reactive Oxygen Species (ROS) metabolism of D-BHB for radiation protection on ground and in Space. General principles and the basic mechanism for ketone supplementation radiation protection has been introduced in an Oxford University publication entitled Ketogenic Diet and Metabolic Therapies: Expanded Roles in Health and Disease (DOI: 10.1093/med/9780190497996.001.0001) chapter Mitigation of Damage from Reactive Oxygen Species and Ionizing Radiation by Ketone Body Esters. As stated and reflected in FIG. 23, “In order to explore KE effects on radiation bone marrow suppression and impact on hematopoiesis, an important mechanism for radiation-induced mortality at the 6 Gy level, the ratio between total reticulocytes and erythrocytes in bone marrow was determined. Bone marrow was extracted as before, 24 hours after KE or saline gavage, a total of 48 hours after either 0.5 or 6 Gy .-radiation. This ratio, which is approximately 1 in bone marrow under normal circumstances, was significantly decreased by both 0.5 and 6 Gy doses of .-radiation in control animals. This decrease was significantly attenuated by 750 mg/kg KE 24 hours after radiation.” As a planned and intended consequence of the claimed invention, radiation protection conferred by the described pathway is logically quicker and more pronounced when utilizing the natural D-Beta HydroxyButyric acid owing to the lack of need for pre-processing as required before utilization of the so called ‘ketone ester’ synthetic product. Owing to the natural origin and improved tolerability of applicant's D-Beta-HydroxyButyric acid, the claimed invention offers improved and long term radiation protection for individuals subjected to radiation exposure both on earth and in space based applications where gamma radiation exposure is a direct and foreseeable concern. Moreover, as reactive oxygen species play a central role in both radiation damage and aging, both processes can be ameliorated by utilization of the claimed invention to increase the amount of antioxidant, enzymes and the reducing power of the NADP-system.

FIG. 24 is a schematic illustration of the protective role of D-BHB on the skin. Since 2000, the ‘500 Dalton’ rule of skin permeability for therapeutic purposes (The 500 Dalton rule for the skin penetration of chemical compounds and drugs by Jan D Bos DOI: 10.1034/j.1600-0625.2000.009003165.x) has offered great insight into compounds with therapeutic potential. Applications of the claimed D-BHB include skin therapeutic protections, especially since the molecular weight of natural D-BHB at 104.1045 g/mol with 1 Da=1 g/mol places skin applications of natural D-BHB well below the 500 Dalton limitation for skin applications. Protecting mitochondria from UV-induced damage, in particular UVA-generated mtDNA mutations, is important for therapeutic and anti-ageing applications. FIG. 24 details the mechanism of age and environmental related skin damage by increasing reactive oxygen species (ROS). Administration of natural D-BHB to skin as a protective and therapeutic will have greater skin penetrance as it is below 500 Daltons and will directly and consequently reduce ROS as an intended and foreseeable consequence of the claimed invention.

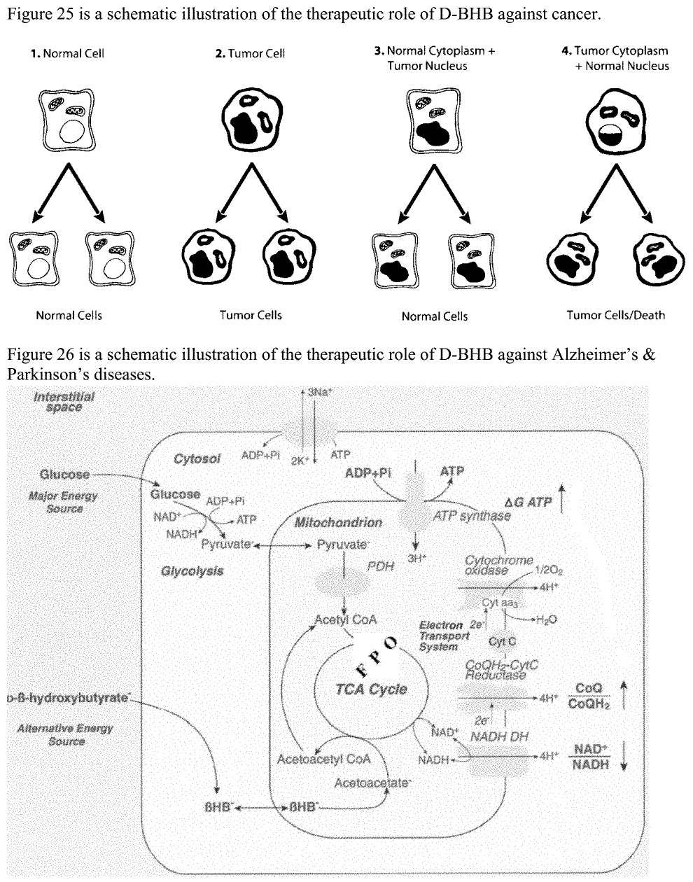

FIG. 25 is a schematic illustration of the therapeutic role of D-BHB against cancer. In Professor Thomas N. Seyfried's seminal work “Cancer as a mitochondrial metabolic disease” (doi: 10.3389/fce11.2015.00043) emerging evidence suggests that cancer is a mitochondrial metabolic disease, according to the original theory of Otto Warburg. FIG. 25 illustrates the role of the nucleus and mitochondria in the origin of tumors. As detailed in the reference, “Normal cells are shown on the left with nuclear and mitochondrial morphology indicative of normal gene expression and respiration, respectively. Tumor cells are shown with abnormal nuclear and mitochondrial morphology indicative of genomic instability and abnormal respiration, respectively. As indicated, (1) Normal cells beget normal cells, (2) Tumor cells beget tumor cells, (3) Transfer of a tumor cell nucleus into a normal cytoplasm begets normal cells, despite the presence of the tumor-associated genomic abnormalities and (4) Transfer of a normal cell nucleus into a tumor cell cytoplasm begets dead cells or tumor cells, but not normal cells. The results suggest that nuclear genomic defects alone cannot account for the origin of tumors, and that normal mitochondria can suppress tumorigenesis.” Therapeutic administration of natural D-BHB is a logical and intended extension of this reasoning. As further observed and described by Otto Warburg, many cancerous tumors can ferment glucose as an energy source but due to mitochondrial damage are incapable of utilizing D-Beta Hydroxybutyric acid as an energy source. When an individual in need thereof utilizes the ketogenic diet in conjunction with the claimed invention, ketone levels can be increased to approach (and in certain cases surpass) glucose levels and as a direct and intended consequence restrict or starve the tumor cell mass from glucose while energizing the body with ketone bodies. Supplementing diet with natural D-Beta Hydroxybutyric acid in conjunction with a ketogenic diet offers improved benefits particularly with respect to hard body cancers such as glioblastoma and breast cancer. For optimum outcomes a Glucose/Ketone Index at or below 1 is optimal in a preferred embodiment.

FIG. 26 is a schematic illustration of the therapeutic role of D-BHB against Alzheimer's & Parkinson's diseases as well as other orphan diseases owing to direct mitochondrial energy supplementation. Studies from early 2001 by NIH's Richard Veech together with Dr. George Cahill entitled “Ketone Bodies, Potential Therapeutic Uses” speculate upon therapeutic potential for Beta HydroxyButyrate against Parkinson's Disease at 4 mmol/L and Alzheimer's Disease at +4.00 mmol/L. The mechanism of action is once again use of BHB as an optimal energy source in the human brain as well as other areas in which there is an energy deficit. As a small molecule, BHB can pass through the blood brain barrier without difficulty. In terms of brain energy it is preferentially metabolized when compared with glucose and unlike glucose does not elicit a toll receptor effect requiring the expenditure of energy in order to offer power to the brain. Similar energy dependent orphan diseases including MADD, Huntington's, Angelmann's syndrome also offer therapeutic opportunities utilizing D-BetaHydroxyButyric acid to increase blood ketone levels according to preferred embodiments of the claimed invention. Preferred embodiments of the claimed invention utilize exogenous D-BetaHydroxy Butyric acid supplementation to overcome the energy deficit presented by these and other related diseases.

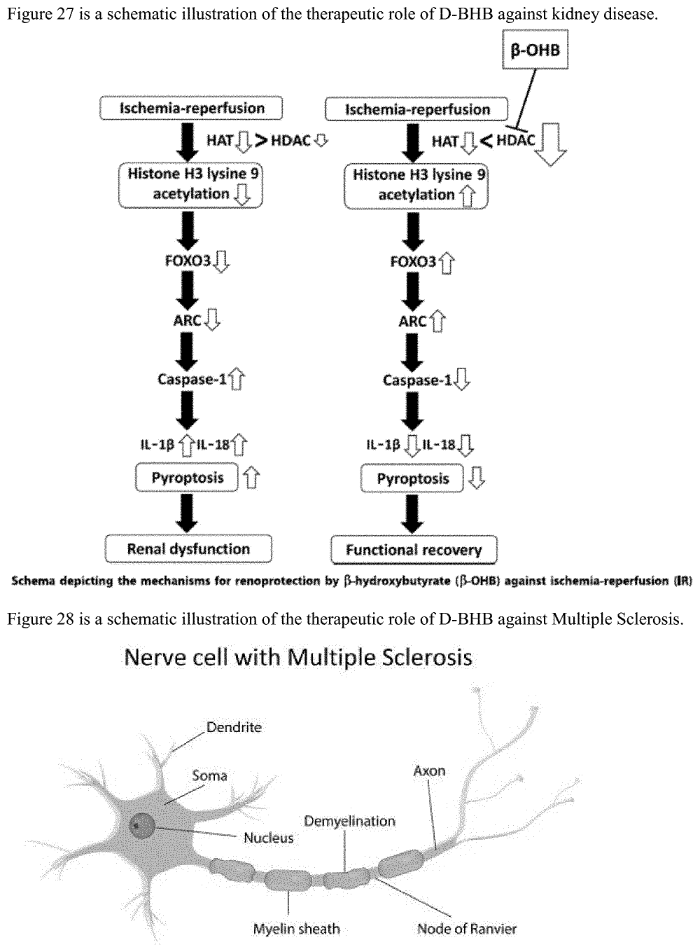

FIG. 27 is a schematic illustration of the therapeutic role of D-BHB against kidney disease. In a related study by Takaya Tajima of Keio University, Tokyo, Japan entitled “b-hydroxybutyrate attenuates renal ischemia-reperfusion injury through its anti-pyroptotic effects” the regulatory effects of Beta Hydroxybutyrate are analyzed with respect to ameliorating kidney damage. While the study appears to have focused on racemic (D+L) sodium salt of Beta Hydroxybutyrate, the example is illustrative of the potential of natural D-BHB as a protectant and therapeutic for kidney damage. FIG. 26 details the distinctive protective regulatory mechanism offered by Beta HydroxyButyrate (BHB) when kidneys are damaged and repaired through BHB administration. As detailed in the reference, “In IR injuries, both histone acetyltransferase (HAT) and histone deacetylase (HDAC) activities decreased although the decrease in HAT activity was more responsible for decreased levels of acetylated H3K9. These changes might downregulate forkhead transcription factor O3 (FOXO3) and apoptosis repressor with caspase recruitment domain (ARC) leading to upregulate caspase-1, interleukin-1b (IL-1b), and IL-18. These gene alterations resulted in pyroptosis and renal tubular injuries after IR insults. The treatment with b-OHB inhibited HDAC activity and further reduced the HDAC activity in the IR-injured kidney and ameliorated the decreased levels of acetylated H3K9. This effect by b-OHB reversed the pyroptosis gene alterations in the IR-injured kidney and ameliorated renal dysfunction. Further evidence for the application of the claimed invention in mitigating kidney disease may be found in the Cell journal publication entitled “Ketosis Ameliorates Renal Cyst Growth in Polycystic Kidney Disease” (doi.org/10.1016/j.cmet.2019.09.012). As a preferred embodiment of the claimed invention, administering a therapeutic amount of natural D-Beta HydroxyButyric acid to achieve a concentration of greater than 3.0 mmol/L in a human offers protective and restorative opportunities when there is a need thereof.

FIG. 28 is a schematic illustration of the therapeutic role of D-BHB against Multiple Sclerosis. Multiple Sclerosis (MS) is a result of an inflammatory and autoimmune driven process which causes damage to the myelin sheath around neurons. This neuron demyelation results in damage to the ability of the nerve cell to propagate electrical signals and make proper connections to other nerve cells. The anti-inflammatory benefits provided by natural D-Beta HydroxyButyric acid supplementation according to the claimed invention offers both signaling and energy benefits provided by the presence of D-BHB to normalize and enhance neuronal function and in a preferred embodiment aid in body improvement over the inflammatory MS condition. In addition, anti-inflammatory benefits against other diseases such as cystic fibrosis are similarly anticipated. In particular, anti-inflammatory benefits of exogenous D-BHB supplementation includes therapeutic treatment against inflammatory diseases with a fibroid basis including polycystic ovarian syndrome (PCOS), endometriosis, pulmonary fibrosis (and by related mechanisms therapies against emphysema) and cystic fibrosis as well as rheumatoid arthritis through inflammasome mediation.

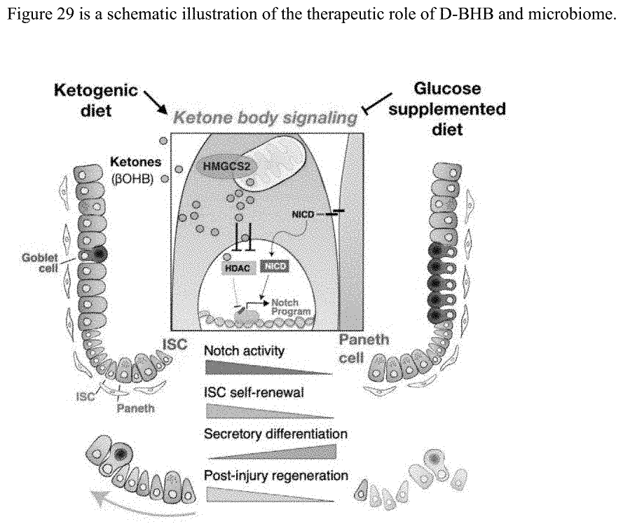

FIG. 29 is a schematic illustration of the therapeutic role of D-BHB and microbiome. In a recent Cell journal article by Professor Cheng entitled “Ketone body signalling mediates intestinal stem cell homeostasis” the signaling aspects of endogenous D-BHB are detailed in FIG. 29. As an intended embodiment of the claimed invention, therapeutic benefits are gained through enhanced stem cell recovery owing to exogenous application of D-Beta HydroxyButyric acid according to the claimed invention.

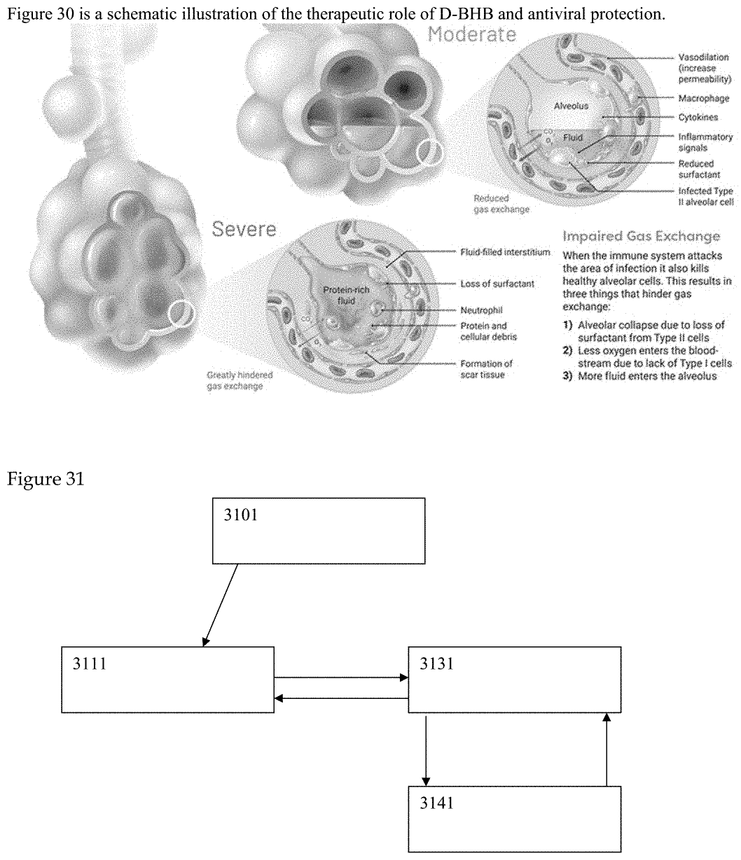

FIG. 30 is a schematic illustration of the therapeutic role of D-BHB and antiviral protection. Attenuation of viral outbreaks are managed through exogenous ketone D-Beta HydroxyButyrate administration owing to the increase in delta gamma T cell production in the alimentary canal and lungs as well as through enhanced oxygen utilization. In acute viral outbreaks such as coronavirus, FIG. 30 details areas of alveolus enhancement due to improved oxygenation in damaged tissue as well as improved immune response. The claimed invention has at least three avenues of attack against viral infections. First, ketone bodies are known to stimulate the Gamma Delta T Cell immune response in the lining of the lungs providing an enhanced physical barrier to infection. Second, exogenous ketone enhancement with natural D-BHB provides increased oxygen utilization in the alveoli owing to the increased presence of and utilization by mitochondria in the alveoli. Third, exogenous D-BHB supplementation provides enhanced kidney protection as detailed previously. In a preferred embodiment, continuous D-Beta HydroxyButyric supplementation at 10 g daily as a prophylactic or 20 g or more as needed is during acute coronavirus attacks is utilized until no longer needed.

FIG. 31 is a schematic illustration of a preferred system embodiment according to the claimed invention. In the exogenous D-BHB ketone administration system, the exogenous natural D-BetaHydroxyButyric acid (3101) is administered to a subject in need thereof (3111) where blood and glucose samples are drawn and administered to measuring test strips (3131) read by a ketone and glucose meter (3141). Blood levels may be monitored by test strips or an in an alternate foreseen embodiment by continuous monitoring devices.

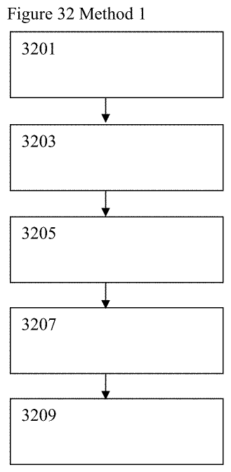

FIG. 32 is a schematic illustration of a preferred method embodiment according to the claimed invention. In a preferred illustrative embodiment, a subject in need thereof measures (3201) endogenous body ketone levels, consumes (3203) exogenous D-Beta HydroxyButyric acid, measures the resultant blood ketone level (3205) after exogenous ketone supplementation, measures the resultant body glucose level (3207) after exogenous ketone supplementation and re-administers (3209) exogenous D-Beta HydroxyButyric acid until the condition for which D-Beta HydroxyButyric acid was required has been mitigated.

In the description, numerous specific details are set forth in order to provide a thorough understanding of the present embodiments. It will be apparent, however, to one having ordinary skill in the art that the specific detail need not be employed to practice the present embodiments. In other instances, well-known materials or methods have not been described in detail in order to avoid obscuring the present embodiments.

Reference throughout this specification to “one embodiment”, “an embodiment”, “one example” or “an example” means that a particular feature, structure or characteristic described in connection with the embodiment or example is included in at least one embodiment of the present embodiments. Thus, appearances of the phrases “in one embodiment”, “in an embodiment”, “one example” or “an example” in various places throughout this specification are not necessarily all referring to the same embodiment or example. Furthermore, the particular features, structures or characteristics may be combined in any suitable combinations and/or sub-combinations in one or more embodiments or examples. In addition, it is appreciated that the figures provided herewith are for explanation purposes to persons ordinarily skilled in the art and that the drawings are not necessarily drawn to scale.

As used herein, the terms “comprises,” “comprising,” “includes,” “including,” “has,” “having,” or any other variation thereof, are intended to cover a non-exclusive inclusion. For example, a process, article, or apparatus that comprises a list of elements is not necessarily limited to only those elements but may include other elements not expressly listed or inherent to such process, article, or apparatus. Additionally, any examples or illustrations given herein are not to be regarded in any way as restrictions on, limits to, or express definitions of any term or terms with which they are utilized. Instead, these examples or illustrations are to be regarded as being described with respect to one particular embodiment and as being illustrative only. Those of ordinary skill in the art will appreciate that any term or terms with which these examples or illustrations are utilized will encompass other embodiments which may or may not be given therewith or elsewhere in the specification and all such embodiments are intended to be included within the scope of that term or terms. Language designating such nonlimiting examples and illustrations includes, but is not limited to: “for example,” “for instance,” “e.g.,” and “in one embodiment.”

I claim:

1. I claim the exogenous ketone D-Beta HydroxyButyric acid prepared as a mendicant for a subject in need thereof at an amount of at least 5 grams D-Beta HydroxyButyric acid.

2. The mendicant of claim 1 additionally comprising a sleep optimized preparation of at least 8 grams D-Beta HydroxyButyric acid.

3. The mendicant of claim 1 additionally comprising an anti-hunger optimized preparation of at least 6 grams D-Beta HydroxyButyric acid.

4. The mendicant of claim 1 additionally comprising an ionizing radiation protection optimized preparation of at least 20 grams D-Beta HydroxyButyric acid.

5. The mendicant of claim 1 additionally comprising an anti-cancer optimized preparation of at least 15 grams D-Beta HydroxyButyric acid.

6. The mendicant of claim 1 additionally comprising an anti-kidney inflammation optimized preparation of at least 18 grams D-Beta HydroxyButyric acid.

7. The mendicant of claim 1 additionally comprising an anti-anxiety optimized preparation of at least 14 grams D-Beta HydroxyButyric acid.

8. The mendicant of claim 1 additionally comprising an anti-Multiple Sclerosis inflammation optimized preparation of at least 24 grams D-Beta HydroxyButyric acid.

9. The mendicant of claim 1 additionally comprising an anti-bone loss optimized preparation of at least 17 grams D-Beta HydroxyButyric acid.

10. The mendicant of claim 1 additionally comprising a heart and oxygen optimized preparation of at least 11 grams D-Beta HydroxyButyric acid.

11. A system of exogenous D-Beta HydroxyButyric acid administration comprising a therapeutic amount of exogenous D-BetaHydroxyButyric acid for administration to a subject in need thereof in conjunction with a means for blood and glucose sample capture in which a subject's blood is drawn and administered to measuring test strips and read by a ketone and glucose meter.

12. A method for exogenous D-Beta Hydroxybutyric acid administration comprising: measuring endogenous body ketone and glucose levels, consuming exogenous D-Beta HydroxyButyric acid, remeasuring the resultant blood ketone and glucose level after exogenous ketone supplementation and re-administering exogenous D-Beta HydroxyButyric acid until the condition for which D-Beta HydroxyButyric acid was required has been mitigated.
